(19) United States
(12) Patent Application Publication
(10) Pub. No.: US 2010/0168774 A1
(43) Pub. Date: Jul. 1, 2010

(54) PRICKING DEVICE

(75) Inventors: Susumu Morita, Osaka (JP); Minoru Yamazaki, Nagano (JP); Hozumi Yoda, Nagano (JP)

Correspondence Address: K&L Gates LLP, P. O. BOX 1135, CHICAGO, IL, 60690, US

(73) Assignee: NISSEI PLASTIC INDUSTRIAL CO., LTD., Nagano (JP)

(21) Appl. No.: 12/645,656

(22) Filed: Dec. 23, 2009

(30) Foreign Application Priority Data
Dec. 25, 2008 (JP) 2008-330679

(51) Int. Cl. A61B 17/34

(52) U.S. Cl. 606/181

(57) ABSTRACT

A pricking device having an accurate pricking function, an automatic safe post-use discarding function and a re-use inhibiting function is disclosed. The pricking device is easy and simple to operate, simple in structure and is high in productivity to reduce production costs. The pricking device is made up of a lancet unit and a housing unit. The lancet unit includes a needle. The lancet unit is formed by a unit body member, a restoration member and a resilient piece for lock molded as one with one another. The unit body member operates to cause the needle to pop out from the housing unit in connection with a pricking operation. The restoration member controls the operation of movement of the unit body member as it stores a force of elasticity in it. After the pricking operation, the needle is returned into the inside of the housing unit under the so stored force of elasticity of the restoration member. Another force of elasticity is stored in the resilient piece for lock as a result of the movement of the unit body member. The resilient piece for lock includes a cam bump engaged with a lock cam section of the housing unit after the pricking operation.

### CROSS REFERENCES TO RELATED APPLICATIONS

The present application claims priority to Japanese Priority Patent Application JP 2008-330679 filed in the Japan Patent Office on Dec. 25, 2008, the entire content of which is hereby incorporated by reference.

### BACKGROUND

The present disclosure relates to a disposable pricking device used for example in sampling a minor quantity of blood from e.g. a finger's tip.

The pricking device is used in a medical site, such as a hospital, a clinic or caring facilities in carrying out an inspection for a blood sugar value for indefinite numbers of patients or test subjects. Such inspection is by sampling minor quantities of blood from e.g. their finger tips. A large variety of the pricking devices are presently sold on the market. The pricking device is required to be used once for all in view of healthcare and from consideration of sanitation. Moreover, since the device has a pricking needle, the device is to be made disposable in safety.

[Patent Publication 1] Japanese Patent Application Publication No. 2007-196010

[Patent Publication 2] Re-published International Patent Application Publication No. WO97/04707

A pricking device, now offered to the market, is made up of a lancet structure and a casing member. The lancet structure is mounted within the casing member for movement therein. The lancet structure is made up of a so-called lancet and a separate resilient member (spring) formed of metal or a resin. The lancet is molded from a single resin material with a pricking needle (needle) buried therein. The separate resilient member is assembled to this lancet either by a manual operation or with an automatic assembling system to complete the lancet structure.

With this type of the pricking device, the spring that forms a part of the lancet structure is formed of metal or resin. With the pricking device, employing the metal spring, such metal spring is desirable to be provided as a separate member, thus incurring the cost for provision and for the spring material. This type of the pricking device is not desirable in case the pricking device is to be made disposable from hygienic reasons.

In addition, with the above sort of the pricking device made up of the lancet structure including the spring and the casing member, not only the number of component parts is increased to raise the production cost, but also the number of assembling process steps is increased to raise the assembling cost. With the above sort of the pricking device, it may be considered to automate the assembling process to lower the assembling cost. However, such automation of the assembling process may be difficult in view of low assembling performance ascribable to the use of a resilient member or of difficulties inherent in the automation of the assembling process.

That is, the resilient member used in a conventional pricking device is in the form of bellows or coils. If larger numbers of these bellows or coils are used, they may be entangled with one another. It is thus necessary to disentangle them into separate parts by a labor-consuming operation. However, in the disentangled state, it is rather difficult to keep the separate parts in the so disentangle state. Moreover, when gripped, the separated bellows or coils tend to be deformed to deteriorate handling or positioning characteristics. Thus, in automatically assembling these resilient components to form a pricking device, the components are desirable to be stored in order in a delivery device annexed to an assembling line. The components, thus stored, are then delivered accurately automatically one by one and assembled in position using e.g. a handling means, by an extremely difficult operation.

The above sort of the pricking device has so far been assembled by a manual operation. This however has presented a problem that productivity may not be improved because of difficulties encountered in handling the resilient members. Since the pricking device is prepared by a manual assembling operation, it is necessary to provide a transfer step as well as a sterilizing or cleaning process between different process steps with consequent rise in production costs. In addition, since the pricking device is larger in size, the production costs are raised in connection with packaging, transport or reservation.

Moreover, with the pricking device employing resilient members in the form of bellows or coils, the pricking operation is carried out against the biasing force of the resilient members. After the end of the pricking, a user releases his/her hand from the lancet structure to retract the pricking needle into the inside of a casing under the force of elasticity stored in the resilient members. The bias force of the resilient members, thus released from the finger pressure, produces an attenuating vibration. However, in the course of the attenuating vibration, the pricking device may prick a plural number of times, specifically, twice or even three times, which is not desirable from the medical point of view.

Therefore, it is desirable to provide a pricking device that has an accurate pricking function, an automatic safe post-use discarding function and a re-use inhibiting function and that is easy and simple to operate, simple in structure and high in productivity to reduce production costs.

### SUMMARY

In one embodiment, a pricking device comprises a lancet unit formed as one with a needle, and a housing that houses the lancet unit for movement therein. The lancet unit includes a unit body member movable within the housing in connection with a pricking operation to cause the needle to pop out of the housing, and a resilient piece. The resilient piece, formed as one with the unit body member, includes a cam bump that may be engaged with a cam groove formed in the housing. The resilient member stores a first force of elasticity as the unit body member is moved in connection with the pricking operation. The cam bump engages within a foremost part of the cam groove when the unit body member has returned into the housing after the pricking operation. The cam bump is kept engaged within the foremost part of the cam groove under the first force of elasticity stored in the resilient piece to keep a state of inhibiting re-popping out of the needle. The unit body member or the housing includes a restoration member that stores therein a second force of elasticity as the unit body member is moved in connection with the pricking operation. The restoration member causes the needle to be returned into the inside of the housing under the second force of elasticity after the pricking operation.

The pricking device according to an embodiment is made up of a lancet unit and a housing. The lancet unit is formed by a unit body member having a needle and a resilient member, which is formed as one with the unit body member. The restoration member is provided on the unit body member or on the housing. The number of component parts is reduced and the structure is simplified to improve the productivity as well as to reduce the device size and costs. In addition, the operation of the device may be simplified. With the pricking device according to an embodiment of the present invention, in which there is provided the restoration member that stores the second force of elasticity in connection with the operation of movement of the unit body member, the unit body member may automatically be returned into the housing member after the pricking operation to provide for improved operability and safety. With the pricking device according to an embodiment of the present invention, in which there is provided the resilient piece that stores the first force of elasticity in connection with the operation of movement of the unit body member. Hence, the unit body member may be retained in the returned state in the housing after the pricking operation. In addition, the re-popping out of the pricking means may be inhibited so that faulty operations may be prevented from occurring to assure improved safety.

Additional features and advantages are described herein, and will be apparent from the following Detailed Description and the figures.

### BRIEF DESCRIPTION OF THE FIGURES

FIG. 1 is a perspective view showing a pricking device of a first embodiment, with a housing unit being shown in a developed state.

FIG. 2 is a perspective view showing the pricking device before use.

FIG. 3 is a perspective view showing a lancet unit of the pricking device.

FIG. 4 is a perspective view showing a housing unit of the pricking device.

FIG. 5 is a front view showing an initial stage of thrusting of an actuation plate, for illustrating the method of using the pricking device.

FIG. 6 is a front view showing a state where a needle is about to be protruded from the housing unit in the course of the pricking operation.

FIG. 7 is a front view showing the state directly following the end of the pricking operation.

FIG. 8 is a front view showing the stage when the pricking operation has been finished and the lancet unit is re-accommodated within the housing unit.

FIG. 9 is a perspective view of a pricking device provided with a lancet pop-out adjustment mechanism according to a second embodiment.

FIG. 10 is a perspective view of a pricking device having a lancet unit according to a third embodiment.

FIG. 11 is a perspective view looking from the reverse side of the main housing unit of the pricking device shown as a third embodiment.

### DETAILED DESCRIPTION

A pricking device 1, shown in the drawings as an embodiment, will now be described in detail with reference to the drawings.

<Global Configuration>

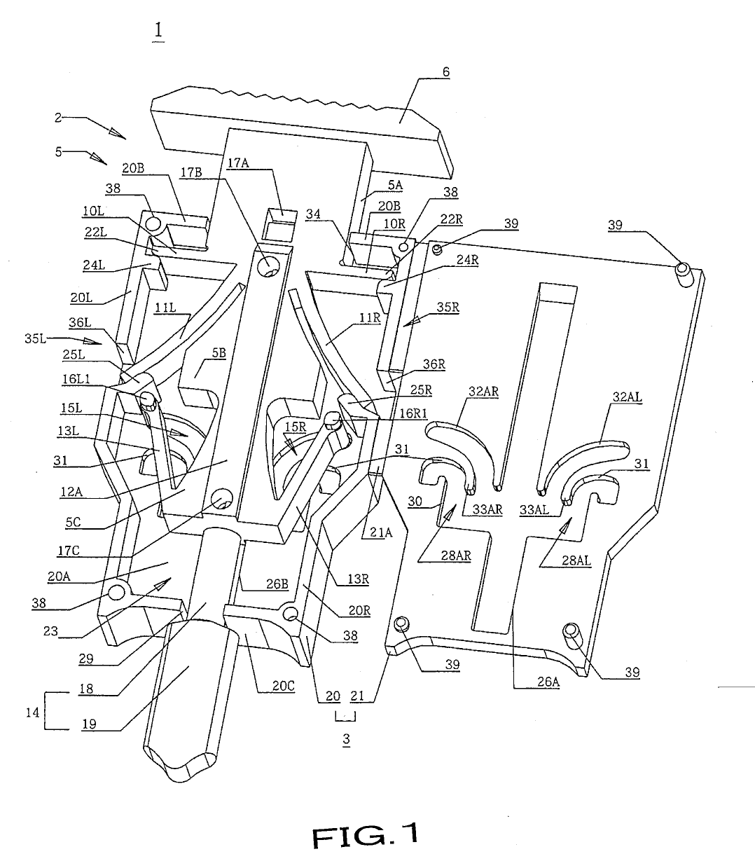

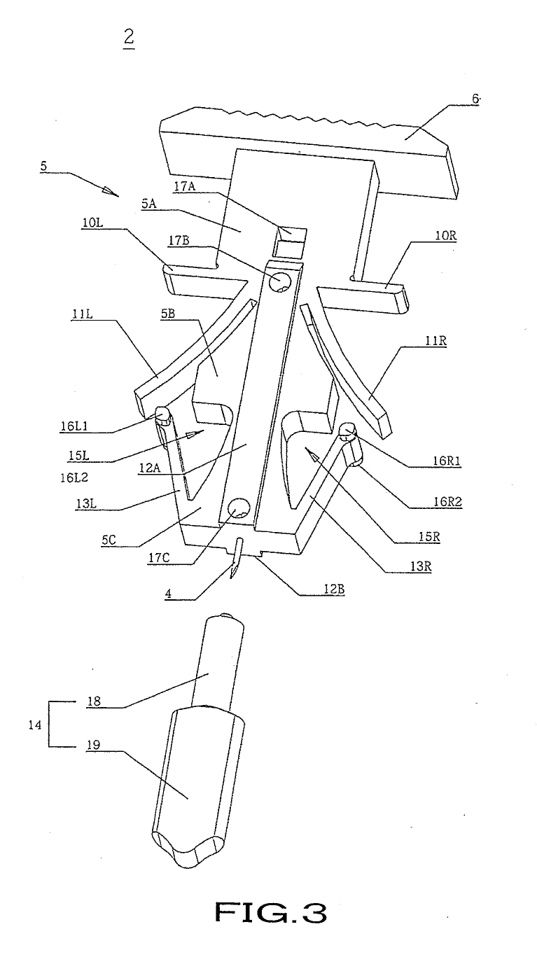

The pricking device 1 is made up of a lancet unit 2 described later in detail and a housing unit 3, as shown in FIGS. 1 to 3. The housing unit 3 is composed of a main housing member 20 and an upper plate 21. For measurement of a blood sugar value or discrimination of a blood type, a needle 4 (see FIG. 3) provided on the lancet unit 2 is popped out of the housing unit 3 to prick the finger's end of a test subject to sample a minor quantity of blood from the so pricked site. In the following description, the expression ‘left’, ‘right’, ‘front’ or ‘reverse’ is to be construed using FIG. 1 as a reference.

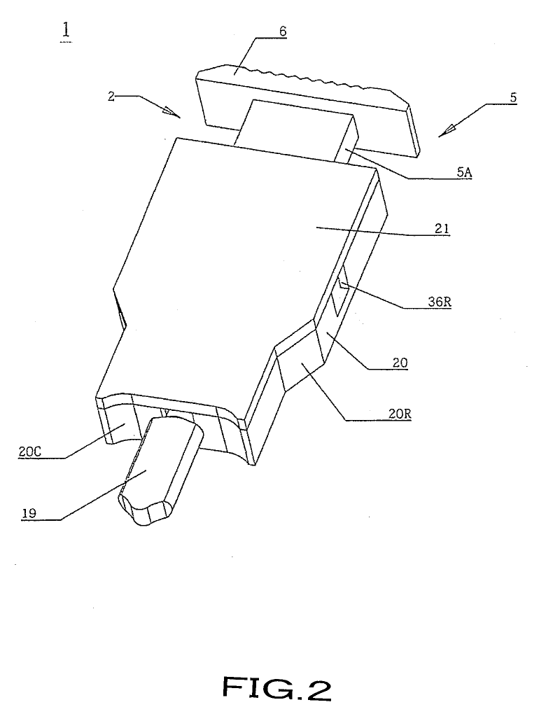

The pricking device 1 presents an appearance shown in FIG. 2. If once the device is used for pricking, the re-popping out of the needle 4 is to be inhibited. Also, the device is to be discarded as the needle 4, once popped out, is housed in a safe condition within the housing unit 3, in a manner which will be explained below in more detail. It should be noted that, although the pricking device 1 shown here by way of an embodiment is of the type in which the needle 4 is popped to prick the finger's end, for example, of the test subject, the present invention is not to be restricted to such device type. For example, the needle 4 may be replaced by a blade that may be protruded out of the housing unit 3 to slightly cut the skin.

With the pricking device 1, the lancet unit 2 excluding the needle 4, the main housing member 20 of the housing unit 3, and the upper plate 21, are molded as respective separate components from synthetic resin materials. These synthetic resin materials may be exemplified by L-LDPE (low density linear polyethylene resin), POM (polyacetal resin), PBT (polybutylene terephthalate resin), polyamide resin or polycarbonate resin. The pricking device 1 is to be designed and constructed so that its various component parts will perform elastic displacement or sliding movement or will undergo breaking in a manner that will be described subsequently. Moreover, the device has to be sterilized in its entirety. Hence, the pricking device 1 is preferably formed of a material that has high elasticity, high toughness, a low frictional coefficient characteristic and high resistance against the processing for sterilization. Preferably, the lancet unit 2 and the housing unit 3 are molded from L-LDPE and polypropylene, respectively.

<Lancet Unit>

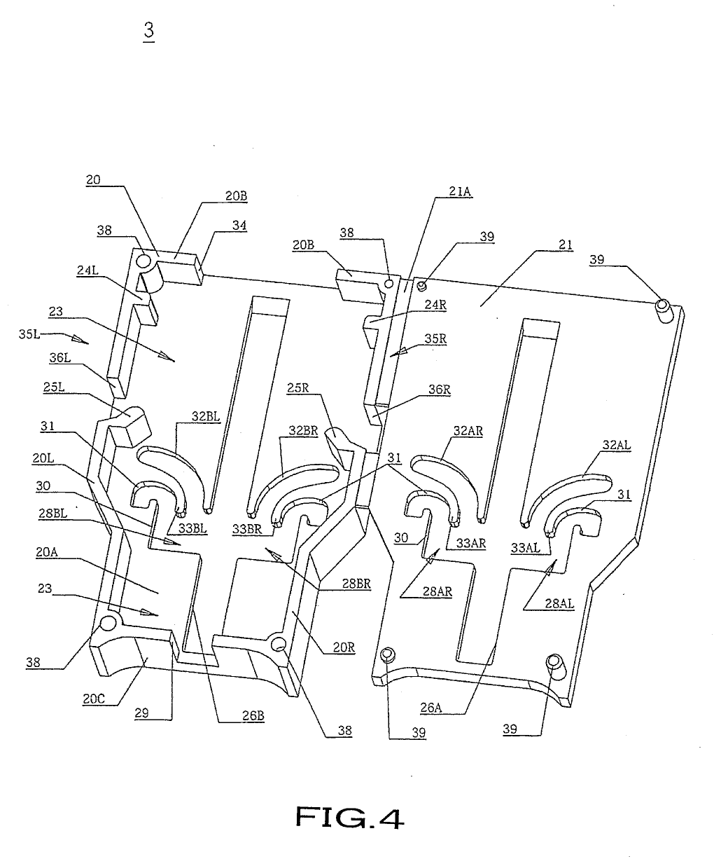

The lancet unit 2 of the pricking device 1 is in the form of a vertically elongated plate having two narrowed-down sites along the height-wise direction (length-wise direction), as shown in FIGS. 1 and 4. Specifically, the lancet unit 2 includes a unit body member 5 made up of an upper part 5A, a mid part 5B and a lower part 5C. The upper part 5A is downwardly arrow-shaped and has its lowermost end delimited by a first narrowed-down section. The mid part 5B, substantially in the form of a hexagon, is contiguous to the lowermost end of the upper part 5A and delimited by the first and second narrowed-down sections. The lower part 5C, substantially in the form of an isosceles triangle, is contiguous to the lowermost end of the mid part 5B, and has its upper end delimited by the second narrowed-down section.

The unit body member 5 forms the basic member that goes to make up the lancet unit 2. In addition, an actuation plate 6 is formed as one with the upper part 5A, and left and right pair unit body member controlling sections 10L, 10R are also formed as one with the upper part 5A. These controlling sections are collectively termed a unit body member controlling section 10, unless they are to be referred to as separate sections. The mid part 5B of the unit body member 5 of the lancet unit 2 is formed as one with left and right pair restoration members 11L, 11R that extend from the site of contiguity of the mid part to the upper part 5A and that act as a first resilient means. These restoration members are collectively termed a restoration member 11 unless they are to be referred to as separate sections.

The lower part 5C of the unit body member 5 of the lancet unit 2 is formed as one with front and reverse pair guide ribs 12A, 12B and left and right pair resilient pieces for lock 13L, 13R. The front and reverse pair guide ribs 12A, 12B are collectively termed a guide rib 12 unless these pair guide ribs are to be referred to as separate sections. The left and right pair resilient pieces for lock 13L, 13R acting as a second resilient means are referred to as a resilient piece for lock 13, unless these pair restoration members are to be referred to as separate sections. The needle 4 is buried as one with and in the bulk of the unit body member 5, as shown in FIG. 3. The needle is so buried in the unit body member 5 for extending along substantially the entire length of the unit body member 5. The needle 4 of the lancet unit 2 is molded as one with and in the bulk of the unit body member 5 so that its lower end is projected out of the lower part 5C of the unit body member 5. The projected portion of the needle 4 is sealed by a lancet cover member 14 formed as one with the unit body member 5.

<Unit Body Member>

The actuation plate 6 of the unit body member 5 is formed as a horizontally elongated rectangular plate shaped member having a cross-section larger than that of the upper end of the upper part 5A. The actuation plate 6 is thrust during the pricking operation as later explained so as to cause downward movement of the unit body member 5 within the inside of the housing unit 3. The actuation plate 6 having a large surface improves the ease in the pricking operation. In addition, the actuation plate 6 abuts against the upper surface of the housing unit 3 to act as a stopper to inhibit an excess thrusting operation. The unit body member 5 is formed with opening windows 17A, 17B and 17C at the mid portions of the upper part 5A, mid part 5B and the lower part 5C, respectively. These opening windows serve for holding the needle 4 and for effecting needle position registration at the time of molding the lancet unit 2.

The unit body member controlling section 10 of the unit body member 5 is formed as one with and protruding from both side edges of the upper part 5A at a preset distance from the actuation plate 6. The unit body member controlling section 10 includes left and right pair controlling resilient pieces 22L, 22R and left and right pair controlling convexed wall sections 24L, 24R. The pair controlling resilient pieces 22L, 22R, provided on the lancet unit side, are collectively termed a controlling resilient piece 22. The left and right pair controlling convexed wall sections 24L, 24R, provided on the housing unit side for facing the controlling resilient piece 22, are collectively termed a controlling convexed wall section 24. Before use of the lancet unit, the controlling resilient piece 22 of the unit body member controlling section 10 is positioned within a lancet accommodating void portion 23 of the housing unit 3, specifically, in a void portion delimited between the inner peripheral surface of an upper wall section 20B of the housing unit 3 and the controlling convexed wall section 24. When the pricking operation has been performed, the controlling resilient piece 22 is resiliently displaced to ride over an end of the controlling convexed wall section 24 so as to be moved to below the controlling convexed wall section 24. As the controlling resilient piece 22 of the unit body member controlling section 10 is disengaged from the state of retention by the controlling convexed wall section 24, there is produced a change in the force of thrust applied to the actuation plate 6 to impart a sense of ‘click’ to a user. The spacing between the unit body member controlling section 10 of the unit body member 5 and the actuation plate 6 prescribes the range of movement of the unit body member 5 in the vertical direction within the housing unit 3 in the initial state.

The lower edge of the upper part 5A of the unit body member 5 is inclined gradually downwards to approach to the center side so as to be contiguous to the mid part 5B via the narrowed-down site. The restoration member 11 comprised of a cantilevered piece is formed as one with and projecting outwardly curvilinearly from the lower edge of the upper part 5A. With the lancet unit 2 housed within the housing unit 3, the restoration member 11 is extended towards the housing unit 3, with the foremost part of the restoration member 11 protruding further outwards than the unit body member controlling section 10. The foremost part of the restoration member 11 rests against guide wall sections 25L, 25R each having a smoothed mating surface. These guide wall sections are collectively termed a guide wall section 25.

When the unit body member 5 is moved downwards to perform the pricking operation as later described, the restoration member 11 has its mid portion resiliently displaced due to its reduced thickness and material characteristics. Thus, a force of elasticity is stored therein to control the movement of the unit body member 5. After termination of the pricking operation, the unit body member 5 is moved upwards, by way of a restoration operation, under the force of elasticity stored in the restoration member 11.

The mid part 5B of the unit body member 5 presents the above mentioned hexagonal shape, and has its lower end contiguously connected via a narrowed-down portion to the lower part 5C. The hexagonal shape is such a one in which the mid part 5B is gradually laterally inclined from the above mentioned connection site of the narrow width in transitioning downwards so as to form an inclined lateral side or edge that faces the restoration member 11. The mid part 5B then forms a vertically extending side edge to form the hexagonal shape. The lower part 5C of the unit body member 5 has its lateral side or edge inclined gradually laterally in transitioning downwards so as to form the above mentioned substantially isosceles triangle. The lower part 5C delimits left and right free void areas for displacement 15L, 15R between it and the mid part 5B to allow for elastic displacement therein of the resilient piece for lock 13. The free void areas for displacement 15L, 15R are collectively termed a free void area for displacement 15, unless they are to be referred to as separate sections.

The unit body member 5 has, at its mid portion, a guide rib 12 comprised of a rectangular projection facing both the front and reverse sides. The guide rib 12, composed of a front side guide rib 12A and a reverse side guide rib 12B, is formed as one with the unit body member 5 for extending between the upper part 5A and the lower part 5C. When the lancet unit 2 is housed within the housing unit 3, the guide rib 12 is engaged within a guide cam groove 26 formed in the housing unit 3 side to permit movement of the unit body member 5 in stability during the pricking operation. The unit body member 5 may be improved in its mechanical strength by forming the guide rib 12 with an increased height.

The resilient piece for lock 13, which is a cantilevered section, is formed as one with the lower side edge of the lower part 5C of the unit body member 5 so that the resilient piece for lock 13 faces the restoration member 11. The resilient piece for lock 13 is extended within the free void area for displacement 15 so that its foremost part will be located laterally outside of the mid part 5B. The foremost part of the resilient piece for lock 13 is formed as one with a front side cam bump 16L1, a front side cam bump 16L2, a reverse side cam bump 16R1 and a reverse side cam bump 16R2. These cam bumps are collectively referred to as a cam bump 16 unless they are to be referred to as separate sections.

When the lancet unit 2 is housed within the housing unit 3, the resilient piece for lock 13 faces a lock cam section 28 provided on the housing unit 3 side in a manner which will be explained subsequently. The cam bump 16 is engaged with the lock cam section 28 to provide an automatic lock mechanism. When the lancet unit 2 is moved in the course of the pricking operation, the resilient piece for lock 13 is moved within the free void area for displacement 15 to store a force of elasticity therein. After the pricking operation, the cam bump 16 is moved to a position of engagement with the lock cam section 28 under the force of elasticity stored in the resilient piece for lock 13. The resilient piece for lock 13 holds the unit body member 5 in a locked state under the force of elasticity stored therein to inhibit the re-pricking operation of the lancet unit 2.

In the lancet unit 2, the needle 4 formed of stainless steel is buried at a mid portion of the unit body member 5. The needle 4 has its upper end located facing the opening 17A in the vicinity of the lower edge of the upper part 5A, while having its lower end protruded a preset length from the lower part 5C. The lancet unit 2 is molded by a so-called insert molding method according to which a synthetic resin material is injected in a molten state into a cavity of a metal mode as the needle is held in place within the metal mold. Since the needle 4 is molded to a sufficient length as one with and in the inside of the unit body member 5, the pricking operation may be carried out in a stabilized state without the risk of producing an offset.

The needle 4 is molded as one with the unit body member 5 so that its foremost part is protruded a preset length out of the housing unit 3 as the lancet unit 2 is moved in the course of the pricking operation. The needle 4, buried for its substantially entire length at mid portions of the lower part 5C and the mid part 5B of the unit body member 5, performs the role of a sort of a reinforcement bar to contribute to high mechanical strength of the unit body member 5.

In the lancet unit 2, as mentioned previously, the protruded portion of the needle 4 from the unit body member 5 is sealed by the lancet cover member 14 formed as one with the unit body member 5. The lancet cover member 14 is made up of a shaft-shaped cover 18 and a grip 19. The shaft-shaped cover 18 seals the needle 4 within the housing unit 3 with the lancet unit 2 housed within the housing unit 3. The grip 19 is protruded from a lancet pop-out opening 29 formed in the housing unit 3, as will be explained subsequently.

In the lancet cover member 14, the shaft-shaped cover 18 is of an axial length approximately equal to the stroke of movement of the lancet unit 2 prescribed by the above mentioned actuation plate 6 and the unit body member controlling section 10. The lancet cover member 14 may readily be broken off by the connecting portion of the shaft-shaped cover 18 connecting to the unit body member 5 being reduced in thickness, for instance. In performing the pricking operation described later, the grip 19 of the lancet cover member 14 may be held by hand and twisted to break the shaft-shaped cover 18 at its connecting portion to the unit body member 5 to expose the needle 4.

The lancet cover member 14 seals the needle 4 until the time of the pricking operation in order to keep the pricking device 1 in a safe condition. As mentioned previously, the lancet unit 2 is molded with the needle 4 in a sterilized state. This sterilized state of the needle 4 is kept until the time of the pricking operation in order to keep the pricking device 1 in a sanitary condition.

<Dimensional Prescriptions>

The dimensional prescriptions of the lancet unit 2 composed of the above mentioned component parts will now be described. It should be noted that the lancet unit 2 is not limited to these dimensional prescriptions. Also, tolerance values are set for the dimensional values. The lancet unit 2 has an overall length of 48.0 mm, with the unit body member 5 being 29.4 mm in length. The unit body member 5 has an overall thickness of 3.0 mm. Since the cam bump 16, 0.8 mm in height, and the guide rib 12, 0.8 mm in height, are formed on the resilient piece for lock 13, the unit body member 5 has a maximum thickness of 4.6 mm. The restoration member 11 has a width of 3.0 mm, a thickness of 0.8 mm and a length of 10.0 mm. The resilient piece for lock 13 is of a width of 3.0 mm, a thickness of 0.8 mm and a length of 9.0 mm.

Referring to FIGS. 1 and 2, the lancet unit 2 constructed as set out above is housed within the housing unit 3 for movement in the up-and-down direction, to constitute the pricking device 1. In this housed state of the lancet unit 2, the actuation plate 6 and a portion of the upper part 5A of the unit body member 5 are protruded from an upper side, while the grip 19 of the lancet cover member 14 is protruded from a lower side. The housing unit 3 is made up of a main housing member 20, an upper plate 21 and a hinge 21A interconnecting the main housing member 20 and the upper plate 21, as shown in FIGS. 1 and 4, and is formed of the synthetic resin as is the above mentioned lancet unit 2.

Meanwhile, the housing unit 3 is not limited to be formed as a unitary member by having the main housing member 20 and the upper plate 21 interconnected by the hinge 21A. For example, the main housing member 20 and the upper plate 21 may be separate molded members without using the hinge 21A.

When the lancet unit 2 of the above mentioned dimensional prescriptions is housed within the housing unit 3, and the main housing member 20 is connected to the upper plate 21 to form a unitary structure of the housing unit 3, the pricking device 1 with a thickness of 6.0 mm, a length of 29.4 mm and a width of 19 mm is formed. When the lancet unit 2 is housed within the housing unit 3 with the grip 19 of the lancet cover member 14 protruded from the main housing member 20 as later explained, the pricking device 1 with an overall length of 48.0 mm is formed. The pricking device 1 is smaller in size and thickness than the above mentioned conventional pricking device. The pricking device 1 outperforms the above mentioned conventional pricking device as to handling such as packaging or transport and as to reduction of costs inclusive of those for management and preservation.

<Housing Unit 3>

The main housing member 20 is formed to a substantially rectangular box of a reduced thickness. It has a bottom plate 20A and outer peripheral wall sections, comprised of an upper surface wall section 20B, a lower surface wall section 20C, a left side wall section 20L and a right side wall section 20R. These peripheral wall sections are formed for upstanding from and extending along the outer peripheral sides of the bottom late 20A. These outer peripheral wall sections of the main housing member 20 are of a height slightly larger than the above mentioned thickness of the lancet unit 2. Thus, the main housing member 20 forms, in conjunction with the upper plate 21, the lancet accommodating void portion 23 within which the lancet unit 2 may be housed for movement with a certain allowance along the direction of thickness. The lower surface wall section 20C of the main housing member 20 is slightly arched along the left-and-right direction to conform to the shape of the finger's tip of a test subject. The position of the pricking device 1 may thereby be kept in a stable condition at the time of pricking of the finger's tip.

At a center portion of the bottom plate 20A of the main housing member 20, there is formed a guide cam groove 26B having a length corresponding to the entire length in the height-wise direction. The guide cam groove 26B forms a guide cam groove 26 in conjunction with a guide cam groove 26A formed in the upper plate 21 side. The guide cam grooves 26A, 26B are collectively termed the guide cam groove 26 unless they are referred to as separate sections.

When the lancet unit 2 is housed within the housing unit 3, the reverse surface side guide rib 12B formed on the unit body member 5 is engaged in the guide cam groove 26B, whilst the front surface side guide rib 12B is engaged in the guide cam groove 26A. The guide cam groove 26B is formed to a groove width slightly larger than the width of the guide rib 12B and a length (height) larger than the height of the guide rib 12B. Thus, in the pricking operation, the guide rib 12B is allowed to be moved downwards to enable the lancet unit 2 to be moved in stability.

The lock cam section 28 that forms the automatic lock mechanism is formed on both sides of the above mentioned guide cam groove 26B of the bottom plate 20A of the main housing member 20. The lock cam section locks the resilient piece for lock 13 after the pricking operation in cooperation with left and right pair lock cam sections 28AL, 28AR formed on the upper plate 21 side as later described.

The lock cam section 28 is formed of left and right pair lock cam sections 28BL, 28BR. These lock cam sections 28BL, 28BR and lock cam sections 28AL, 28AR are collectively termed the lock cam section 28 unless they are to be referred to as separate sections.

In the lock cam section 28, when the lancet unit 2 is housed within the housing unit 3 following the end of the pricking operation, the reverse side cam bumps 16L2, 16R2, formed on the reverse side of the unit body member 5, are engaged with the lock cam sections 28BL, 28BR, while the front side cam bumps 16L1, 16R1, formed on the front side of the unit body member 5, are engaged with the lock cam sections 28AL, 28AR. With the lock cam section 28, the lock cam sections 28AL, 28AR are of the same shape as the lock cam sections 28BL, 28BR. In addition, the lock cam sections are in left-right symmetry with respect to each other. Hence, only the sole lock cam section 28 is described with the use of reference numerals used in common with the remaining lock cam sections, and the description of these remaining lock cam sections is dispensed with.

Referring to FIGS. 1 and 4, the lock cam section 28 includes a substantially rectangular cam opening 30, a cam recess 31, a cam groove 32, and a guide cam tongue 33. The cam opening 30 is formed in the bottom plate 20A of the main housing member 20 and in the upper plate 21 to a depth that allows for engagement with the cam bumps 16L, 16R. The cam recess 31 and the cam groove 32 are contiguous to the cam opening 30. The guide cam tongue 33 separate the cam recess 31 and the cam groove 32 from each other. In the lock cam section 28, with the lancet unit 2 housed within the housing unit 3, the cam opening 30 faces the proximal end of the resilient piece for lock 13 of the unit body member 5, as shown in FIG. 1. The cam opening 30 is formed as a substantially rectangular opening.

The guide cam tongue 33 is substantially hook-shaped and projected towards the inner side from a mid portion of the upper edge of the cam opening 30 in the lock cam section 28, as shown in FIG. 4. The cam recess 31 and the cam groove 32 are formed towards the lateral side and towards the center of the guide cam tongue 33. The cam groove 32 of the lock cam section 28 is formed as an inclined cam groove that is progressively laterally inclined towards its foremost part. With the lancet unit 2 housed within the housing unit 3, the cam bump 16 formed on the unit body member 5 is engaged in the cam groove 32. In an initial state, the cam bump 16 is engaged in the foremost part of the cam groove 32 of the lock cam section 28.

As the lancet unit 2 is moved downwards within the housing unit 3 to perform the pricking operation, the lock cam section 28 causes the cam bump 16 to be moved away from the foremost part of the cam groove 32 into the cam opening 30 to cause resilient displacement of the resilient piece for lock 13. During the pricking operation, the lock cam section 28 retains the cam bump 16 in the state of engagement in the cam opening 30. This causes a force of elasticity to be stored in the resilient piece for lock 13.

After the pricking operation, the lock cam section 28 produces the operation of restoration of the resilient piece for lock 13 under the force of elasticity stored therein. At this time, the cam bump 16 is moved away from the cam opening 30 so as to be engaged with the cam recess 31 under the action of the guide cam tongue 33, so that the resilient piece for lock 13 is held at the foremost part of the cam recess 31.

The cam recess 31 is formed in the bottom plate 20A of the main housing member 20 and in the upper plate 21, as shown in FIGS. 1 and 4. The cam recess 31 has its proximal part contiguous to the cam opening 30, while having its foremost part arched towards the lateral side. As the cam bump 16 is moved away from the cam opening 30 into the cam recess 31 to get to the foremost part of the cam recess 31, the cam bump 16 of the resilient piece for lock 13 is engaged in the cam recess 31 to lock the resilient piece for lock 13. After the pricking operation, the cam recess 31 locks the resilient piece for lock 13 in cooperation with the lock cam section 28. Hence, a re-pricking operation, attempted inadvertently, may be inhibited.

A guide hole 34 is formed in the upper surface wall section 20B of the main housing member 20 facing the upper end of the guide cam groove 26B. The guide hole 34 is larger in cross-section area than the upper part 5A of the unit body member 5 and smaller in size than the outer dimension of the actuation plate 6 or the unit body member controlling section 10. The actuation plate 6 is protruded outwards as the lancet unit 2 is housed in the lancet accommodating void portion 23 via this guide hole 34 formed in the main housing member 20.

In performing the pricking operation as later described, the guide hole 34 in the main housing member 20 controls the upper part 5A of the unit body member 5 to cause movement of the lancet unit 2 in stability. The lancet pop-out opening 29 is formed in the lower surface wall section 20C of the main housing member 20 facing the lower end of the guide cam groove 26B.

The lancet pop-out opening 29 is formed to an opening size larger than the cross-section area of the shaft-shaped cover 18 of the lancet cover member 14 and smaller than that of the grip 19. The lancet pop-out opening 29 is formed at an uppermost site of the arched portion of the lower surface wall section 20C of the main housing member 20, that is, a mid portion in the upper surface wall section 20B. The lancet unit 2 is housed within the lancet accommodating void portion 23 as the proximal end of the grip 19 abuts against the outer surface of the lower surface wall section 20C and the shaft-shaped cover 18 is fitted in the lancet pop-out opening 29, as shown in FIG. 1. The lancet cover member 14 is broken off at the grip 19 protruded at the time of the pricking operation, so that the foremost part of the needle 4 exposed from the unit body member 5 will pop out via the lancet pop-out opening 29.

<Resilient Piece Controlling Section>

The main housing member 20 is formed as one with resilient piece controlling sections 35L, 35R, which are disposed at opposite positions of the left side wall section 20L and the right side wall section 20R for facing the restoration member 11. These resilient piece controlling sections are collectively termed a resilient piece controlling section 35 unless they are referred to as separate sections. The resilient piece controlling sections are of the same symmetrical shape. Hence, only the resilient piece controlling section 35L provided on the left side wall section 20L is described with reference to FIG. 4 with the use of reference numerals used in common with the resilient piece controlling section 35L.

The resilient piece controlling section 35 is made up of a guide opening 36 formed in the left side wall section 20L and a guide wall section 25 bent inwards at a lower opening edge of the guide opening 36. With the lancet unit 2 housed in the housing unit 3, the foremost part of the restoration member 11, formed on the unit body member 5, faces the guide opening 36, as shown in FIG. 1.

The guide wall section 25 of the resilient piece controlling section 35 is an upstanding wall section slightly inclined towards the upper surface wall section 20B. An inner surface of the foremost part of the wall section is substantially arcuate in shape. The guide wall section 25 of the resilient piece controlling section 35 has a smooth receiving surface for extending substantially along the extending direction of the restoration member 11.

The guide wall section 25 of the resilient piece controlling section 35 is in sliding contact with the inner lateral side of the restoration member 11 and thus acts as a receiving surface. When the lancet unit 2 has moved more than a preset value, the resilient piece controlling section 35 allows the foremost part of the restoration member 11 to escape towards outside of the left side wall section 20L via the guide opening 36. The resilient piece controlling section 35 is designed and constructed so that the force of elasticity stored in the restoration member 11 acts as a driving force to produce the restoration operation for the lancet unit 2 following the pricking operation.

At the corners of the outer peripheral wall sections of the main housing member 20, there are formed fitting holes 38. In these fitting holes 38, fitting pins 39 provided on the upper plate 21 are engaged to unify the main housing member 20 and the upper plate 21 together. Of course, the fitting holes 38 do not have to be formed at the respective corners. For example, these fitting holes may be formed on the upper surface wall section 20B in clipping the guide hole 34 as large-sized holes. Or, the fitting holes may be formed at optional sites of the resilient piece controlling section 35 on which the force of elasticity of the restoration member 11 directly acts.

With the above mentioned configuration of the main housing member 20, the hinge 21A is bent, as the lancet unit 2 is housed within the lancet accommodating void portion 23, whereby the upper plate 21 closes the lancet accommodating void portion 23 to unify the housing unit and the lancet unit to each other. This state is referred to below as a unified state. As shown in FIGS. 1 and 4, the upper plate 21 is of the overall planar shape equivalent to the outer shape of the main housing member 20. The upper plate 21 includes the guide cam groove 26A in the height-wise direction corresponding to the guide cam groove 26B of the main housing member 20, while also including the first lock cam sections 28AL, 28AR corresponding to the second lock cam sections 28BL, 28BL.

In the unified state, the upper plate 21 causes the guide rib 12A of the lancet unit 2 to be engaged in the guide cam groove 26A. In the unified state, the cam bumps 16L1, 16R1 on the lancet unit 2 are engaged with the lock cam sections 28AL, 28AR of the upper plate 21.

At the respective corners on the inner side of the upper plate 21, there are formed the fitting pins 39 facing to the fitting holes 38 formed in the main housing member 20. When the upper plate 21 is turned over the main housing member 20 by bending at the hinge 21A, the fitting pins 39 are press-fitted in the mating fitting holes 38, whereby the upper plate 21 may be unified with the main housing member 20. The fitting holes 38 and the fitting pins 39 may all be of the same shape, or may differ in shape from one fitting position to the next, as shown in FIG. 4.

The fitting pins 39 may be in the form of round bars or cylinders if so desired.

With the pricking device 1, the above mentioned lancet unit 2 and the housing unit 3 are molded using respective metal molds designed to fabricate one-piece molded products. The so fabricated lancet unit 2 and housing unit 3 are delivered to, for example, an automatic assembling process to complete the pricking device 1. The metal molds used for shaping the lancet unit 2 or the housing unit 3 are slightly more complex in structure than the conventional metal molds. It is however possible to mold the lancet unit 2 or the housing unit 3 of higher precision more efficiently.

The lancet unit 2 and the housing unit 3 of the pricking device 1 are substantially plate-shaped members, and hence may not be bulky in size or may not be entangled, thus providing for facilitated handling during the molding and assembling processes. In addition, the lancet unit 2 or the housing unit 3 may be placed side-by-side on an automatic transfer machine. It is thus possible to manufacture the pricking device 1 at a drastically reduced cost.

During an assembling process for the pricking device 1, the housing unit 3, supplied from the transfer machine into an assembling cavity of an automatic assembling system, is set with the lancet accommodating void portion 23 of FIG. 4 directed upwards. The lancet unit 2 supplied from another transfer machine is assembled to this housing unit 3. The assembling process may be carried out by dropping the lancet unit 2 into the lancet accommodating void portion 23 to assemble the lancet unit 2 to the main housing member 20 of the housing unit 3.

In the assembling process, the upper plate 21 is placed over the main housing member 20, into which the lancet unit 2 has been dropped. In the assembling process, the upper plate 21 placed on the main housing member 20 is thrust to fit the fitting pin 39 into the fitting hole 38 to assemble the housing unit 3. This completes the pricking device 1, with the actuation plate 6 projecting upwards and with the lancet cover member 14 projecting downwards, as shown in FIG. 2.

The pricking device 1 is small in size, in comparison with the above mentioned conventional pricking device. In addition, it is of a simplified shape like a box of thin thickness, as shown in FIG. 2. The pricking device 1 may readily be packed, while space saving may be achieved in storage or transportation.

<Method for Use>

With the above mentioned pricking device 1, the lancet cover member 14 protruded from the housing unit 3 is broken at the time of the pricking operation. A user holds the housing unit 3 of the pricking device 1 with one hand, and holds and twists the grip 19 of the lancet cover member 14 with the other hand. This breaks the base end of the shaft-shaped cover 18 from the unit body member 5 so that the lancet cover member 14 may be taken out from the housing unit 3.

With the pricking device 1, the foremost part of the needle 4, insert-molded in the bulk of the shaft-shaped cover 18 of the lancet cover member 14, is now exposed. With the pricking device 1, the foremost part of the needle 4 thus exposed is accommodated within the housing unit 3, and hence there is no risk that the finger's tip, for example, of the user or the test subject is inadvertently injured.

With the pricking device 1, the user holds the mid sites of the front and reverse major surfaces of the housing unit 3 with his/her thumb and middle finger, and thrusts its bottom, as the lancet pop-out opening 29 is aligned with the pricking site, such as the finger's tip. With the pricking device 1, the user thrusts the actuation plate 6 protruded on top of the upper plate 21 with his/her trigger finger, whereby the foremost part of the needle 4 pops out via the lancet pop-out opening 29 to prick the finger's tip, for example. Since the lower surface wall section 20C of the main housing member is arched, it fits to a curved surface, such as that of the finger's tip, thus preventing offsetting of the pricking position.

The needle 4 may be housed within the housing unit 3 by the user moves the pricking device 1 away from the pricking site and releasing his/her finger's tip, so far thrusting the actuation plate 6. With the pricking device 1, the lancet unit 2, resiliently held in place within the lancet accommodating void portion 23, is locked by the resilient piece for lock 13. The pricking device 1 is discarded as the needle 4 is housed within the housing unit 3.

<Pricking Operation>

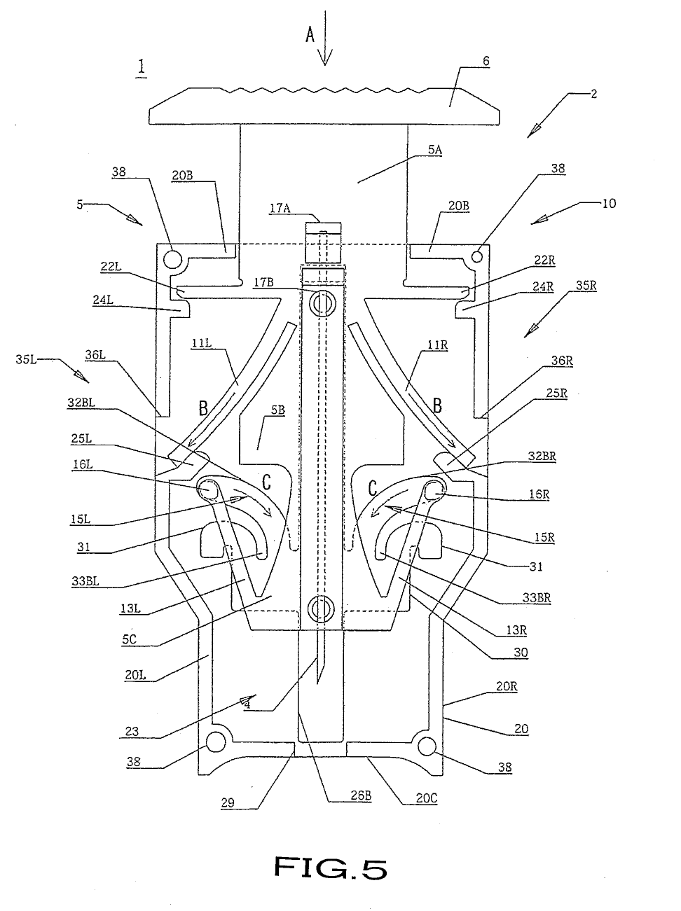

The movement of the lancet unit 2 within the housing unit 3 accompanying the above mentioned pricking operation is now described with reference to FIGS. 5 to 8. When the lancet cover unit 14 is broken off, the needle 4 may have its foremost part exposed from the unit body member 5, as shown in FIG. 5. However, the needle 4 is housed within the housing unit 3. With the pricking device 1, the controlling resilient piece 22 of the lancet unit 2 is situated in a space between the controlling convexed wall section 24 and the upper surface wall section 20B of the main housing member 20. This prescribes the initial position of the pricking device 1.

In the initial position of the lancet unit 2 of the pricking device 1, the foremost part of the restoration member 11 is abutted against the guide wall section 25 of the resilient piece controlling section 35 formed in the main housing member 20. Moreover, in the initial position of the lancet unit 2 of the pricking device 1, the cam bump 16, formed at the foremost part of the resilient piece for lock 13, is engaged with a foremost part of the cam groove 32 of the lock cam section 28 formed at each of the upper plate 21 and the main housing member 20 of the housing unit 3. From this initial position of the pricking device 1, the actuation plate 6 is thrust, as indicated by arrow A in FIG. 5.

With the pricking device 1, the unit body member 5 formed as one with the actuation plate 6 is moved downwards within the housing unit 3. With the pricking device 1, the guide ribs 12, formed on the front and reverse surfaces of the unit body member 5, are moved within the guide cam grooves 26 formed in the main housing member 20 and in the upper plate 21, as the guide ribs are kept engaged with the guide cam grooves 26. The lancet unit 2 may thus be moved downwards in a stabilized position.

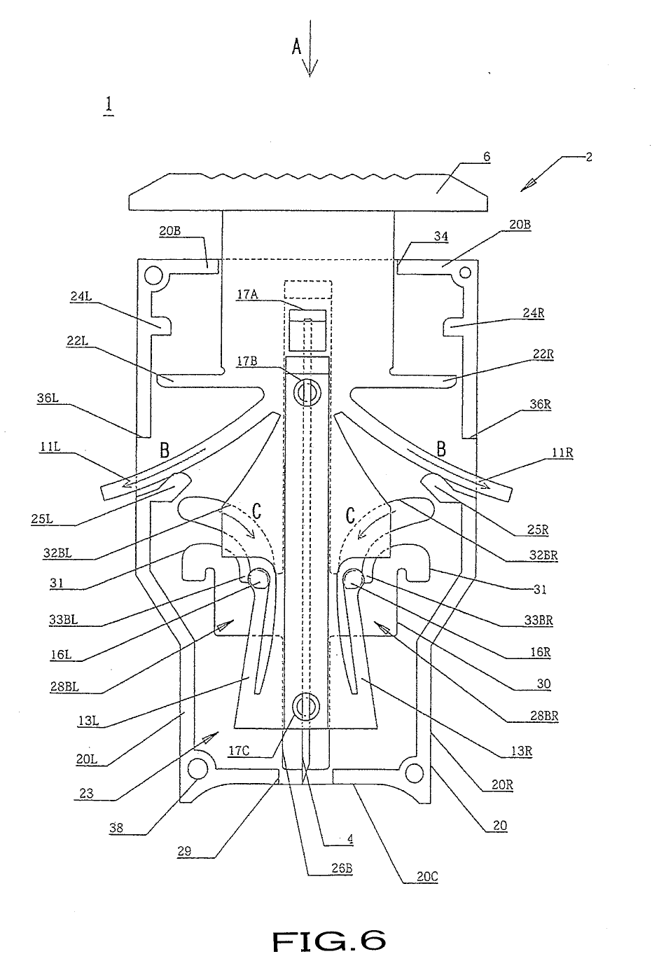

As the actuation plate 6 is thrust and the lancet unit 2 is moved downwards, the restoration member 11 is guided along the guide wall section 25, with the foremost part of the restoration member 11 sliding on the smoothed receiving surface. The foremost part of the restoration member 11 is protruded outwards from the guide opening 36, as shown by arrows B in FIGS. 5 and 6. The force of elasticity is thus gradually stored in the restoration member 11. As the lancet unit 2 of the pricking device 1 is moved further, the cam bump 16 is moved downwards within the cam groove 32, as indicated by arrows C in FIGS. 5 and 6. The resilient piece for lock 13 is gradually elastically displaced within the free void area 15 towards the center of the unit body member 5 to store the force of elasticity therein. As the lancet unit 2 is moved further downwards, the restoration member 11 is bent significantly. On the other hand, the controlling resilient piece 22 of the unit body member controlling section 10 abuts on the controlling convexed wall section 24 and is thereby displaced elastically.

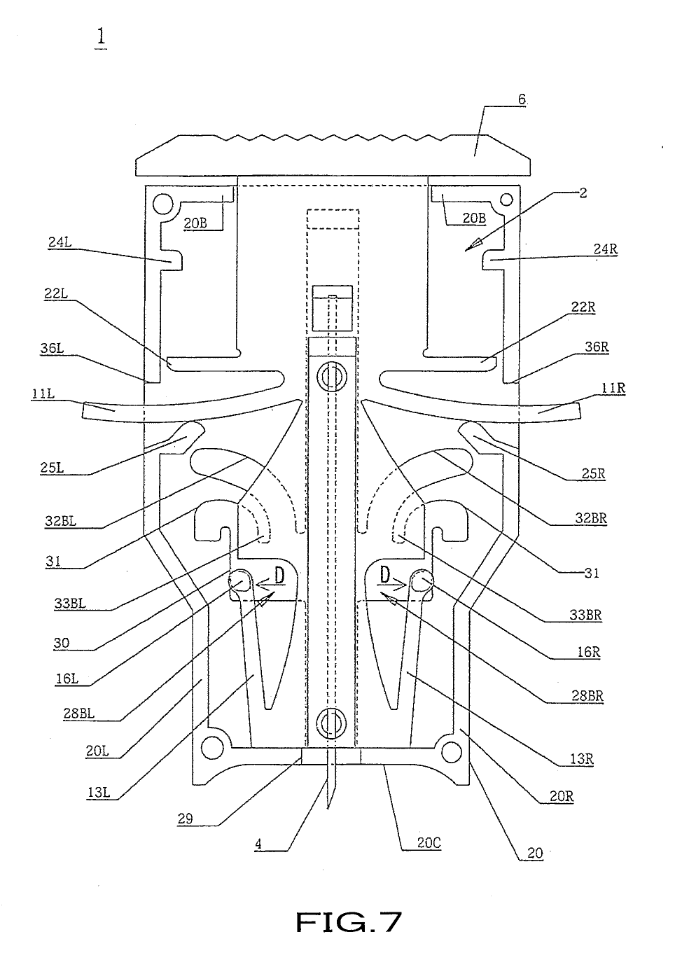

When the unit body member 5 is moved by more than a preset amount, the controlling resilient piece 22 is set free from the controlling convexed wall section 24. Hence, the unit body member 5 is moved vigorously within the housing unit 3, so that the needle 4 is able to pop out from the state shown in FIG. 6 to the state shown in FIG. 7.

The pricking device 1 is halted with the unit body member 5 abutting against the inner surface of the lower surface wall section 20C of the main housing member 20, as shown in FIG. 7. At this time, the foremost part of the needle 4 is protruded a preset length out of the lancet pop-out opening 29. As the lancet unit 2 is moved, the cam bump 16 formed on the resilient piece for lock 13 is moved from the lower end of the cam groove 32 to the cam opening 30 to release a part of the force of elasticity stored in the resilient piece for lock 13. With the resilient piece for lock 13 thus decompressed, the cam bump 16 is moved laterally within the cam opening 30, as indicated by arrow D in FIG. 7.

In an initial stage of thrusting of the actuation plate 6, the lancet unit 2 of the pricking device 1 is restricted its movement. When a preset thrust position is surpassed, the lancet unit 2 performs a movement of descent instantaneously. Hence, the pricking device 1 allows for a stabilized needle pop-out operation. In addition, with the pricking device 1, there is no risk that the lancet unit is vibrated vertically due to variations in the force of elasticity of the elastic member, thus possibly causing pricking a second time or even a third time, as is often the case with the conventional pricking devices.

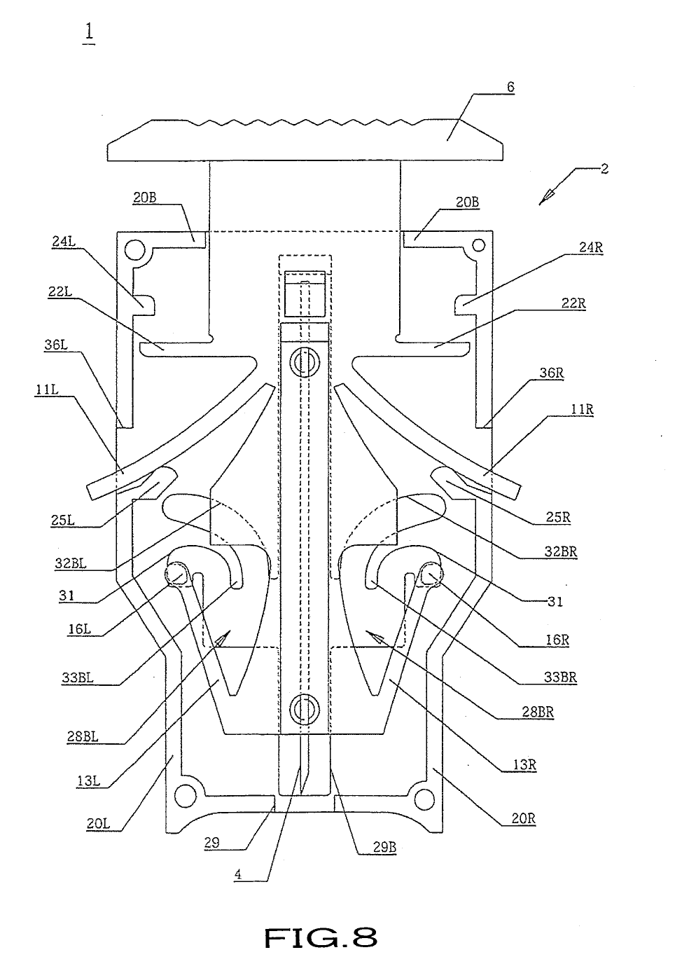

With the pricking device 1, when the user has released his/her trigger finger that has so far thrust the actuation plate 6, there occurs the movement of restoration of the lancet unit 2 as shown in FIG. 8. The unit body member 5 of the pricking device 1 is at a lower position within the housing member 20, and the restoration member 11 is hoisted by the guide wall section 25 of the resilient piece controlling section 35, as shown in FIG. 7. Hence, the force of elasticity is stored in the restoration member 11. As the lancet unit 2 performs its movement of restoration upwards under the force of elasticity stored in the restoration member 11, the needle 4 is retracted via the lancet pop-out opening 29 into the inside of the housing unit 3 of the pricking device 1.

The lancet unit 2 of the pricking device 1 performs a movement of restoration in a stabilized position as the guide rib 12 is engaged within the guide cam groove 26 and moved in this condition. With the movement of restoration of the lancet unit 2, the foremost part of the restoration member 11 is entrained into the inside of the housing unit 3. With the movement of restoration of the lancet unit 2, the resilient piece for lock 13 is also returned as the cam bump 16 formed at its distal end is kept in abutment with the side edge of the cam opening 30 that forms the lock cam section 28 provided on the housing unit 3.

When the lancet unit 2 has been returned to a preset position, the cam bump 16 is engaged in the cam recess 31 of the lock cam section 28. At this time, the cam bump 16 of the resilient piece for lock 13 is engaged in the cam recess 31 formed in the housing unit 3. The cam bump 16 is kept engaged in the cam recess 31 under the force of elasticity stored in the resilient piece for lock 13.

With the pricking device 1, the position of restoration of the lancet unit 2 is prescribed by a state in which the actuation plate 6 has been slightly thrust downwards from the initial position, as shown in FIGS. 5 and 8. In this state, the foremost part of the restoration member 11 is slightly protruded from the resilient piece controlling section 35, with the needle 4 being housed within the housing unit 3.

In this state, the cam bump 16 of the resilient piece for lock 13 is engaged with the foremost part of the cam recess 31 formed in the housing unit 3, and is retained at the foremost part under the force of elasticity stored in the resilient piece for lock 13. Thus, even if the actuation plate 6 is re-thrust, the needle 4 is inhibited from popping out a second time, thus assuring a high degree of safety. With the pricking device 1, even if the actuation plate 6 is inadvertently re-thrust, the cam bump 16 of the resilient piece for lock 13 is retained by the foremost part of the cam recess 31 of the housing unit 3 under the force of elasticity of the resilient piece for lock 13.

In the pricking device 1 shown above as an embodiment, respective component parts of the lancet unit 2 or the housing unit 3, formed of synthetic resin materials, may be changed as desired. The unit body member 5 that forms the lancet unit 2, the restoration member 11 or the resilient piece for lock 13 may be of any suitable shape other than that described above. With the pricking device 1, the main housing member 20 and the upper plate 21 are unified together by press-fitting a plurality of the fitting pins 39 into a plurality of the fitting holes 38. If desired to further improve the mechanical strength of the unified portions, ultra-sonic welding, for example, may be applied to the unified portions.

The pricking device 1 includes an automatic lock mechanism in which the resilient piece for lock 13 provided with the cam bumps 16 is formed on the lancet unit 2, while also including a lock cam section 28 on the housing unit 3. The lock cam section 28 includes the cam opening 30, cam recess 31, cam groove 32 and the guide cam tongue 33. The automatic lock mechanism allows for automatic locking of the lancet unit 2.

With the pricking device described above, the lancet unit 2 and the housing unit 3 may be reduced in thickness, such that it is possible to achieve a global thickness on the order of 4.6 mm. With the pricking device 1, the metal molds used for fabricating the lancet unit 2 and the cam housing 3 by molding may be simplified in structure, thus further reducing the production costs.

### Second Embodiment

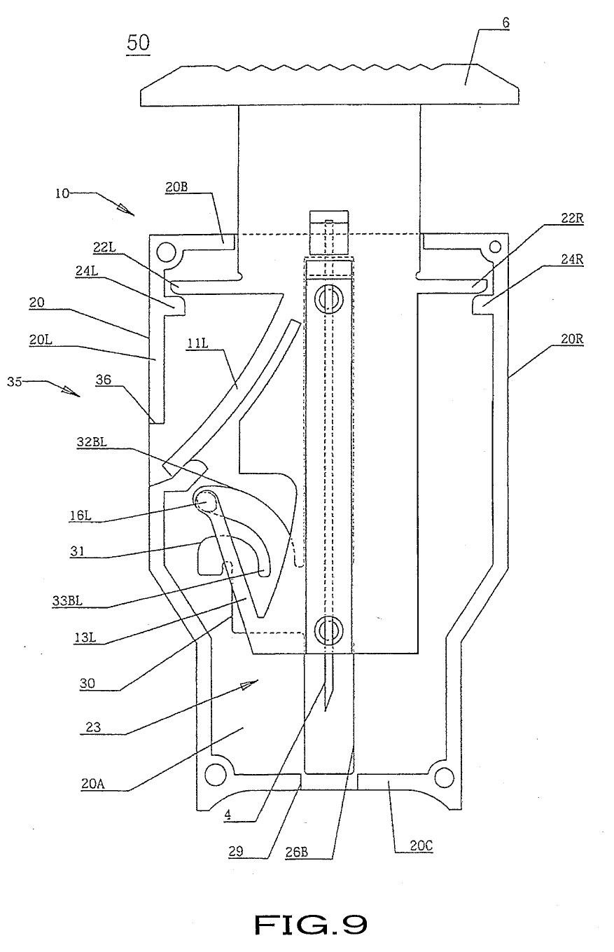

A pricking device 50, shown in FIG. 9 as a second embodiment, has a basic structure in common with the above described pricking device 1. However, with the second embodiment, the structures of the lancet unit 2 and the housing unit 3 are simplified to reduce the size and production costs. Hence, the same reference numerals are used to depict corresponding parts and the description thereof is dispensed with. Stated briefly, with the second embodiment, the resilient piece controlling sections and the automatic lock mechanism for the lancet unit are provided only on one side of the pricking device, that is, on its left or right side, thereby simplifying the structure.

With the pricking device 50, the guide ribs 12 are formed as one with front and reverse side major surfaces of the unit body member 5 of the lancet unit 2, and are engaged in the guide cam groove 26 formed in the main housing unit 20 and in the upper plate 21 for movement in the lancet accommodating void portion 23. The lancet unit 2 is molded integrally of the above mentioned synthetic resin material, while the housing unit 3 is also molded integrally of the above mentioned synthetic resin material, so that component parts of the pricking device 50 may be formed to preset dimensional accuracy and preset mechanical strength. With the pricking device 50, the lancet unit 2 may be moved in stability within the housing unit 3 during the pricking operation. With the pricking device 50, the lancet unit 2, returned into the inside of the housing unit 3, may be kept retained by the lancet unit automatic lock mechanism to inhibit the re-use of the lancet unit.

### Third Embodiment

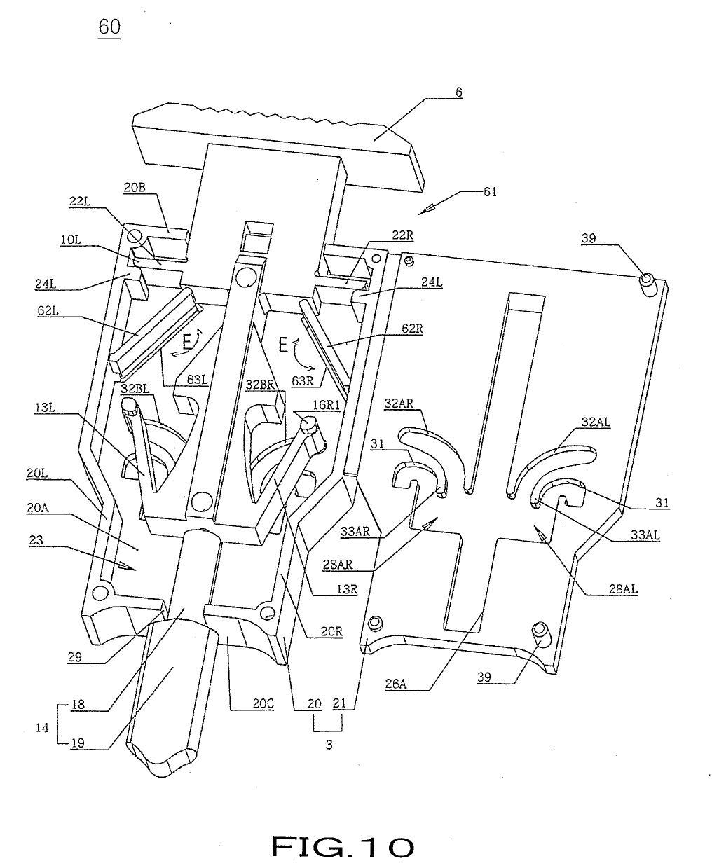

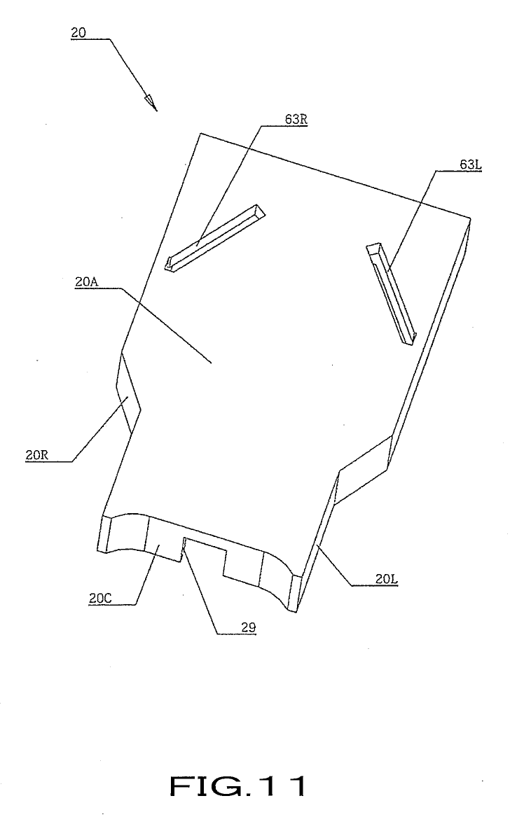

A pricking device 60, shown as a third embodiment in FIGS. 10 and 11, has its basic structure in common with the above mentioned pricking device 1. However, with the present embodiment, a restoration member 11 is provided on the housing unit 3, instead of on the lancet unit 2. Since the pricking device 60 has a basic structure in common with the above mentioned pricking device 1, corresponding parts are denoted by the same reference numerals, and detailed description thereof is here dispensed with.

With the pricking device 60, a lancet unit 61 is used in place of the lancet unit 2, as shown in FIG. 10. This lancet unit 61 is housed within a main housing unit 20. The restoration member 11 provided at the mid part 5B of the unit body member 5 with the above mentioned embodiments is not provided on the lancet unit 61 of the pricking device 60, so that the mid part 5B is simplified in structure. With the pricking device 60, restoration members 62L, 62R are provided on the main housing unit 20 at corresponding positions. That is, the proximal ends of the restoration members 62L, 62R are formed as one with mid portions of left and right side wall sections 20L, 20R of the main housing unit 20, while the foremost parts thereof abut against the lower end of the upper part 5A. The restoration members 62L, 62R may be moved as indicated by arrow E in FIG. 10, so that there is stored a force of elasticity therein with downward movement of the lancet unit 61.

Since the pricking device 60 is provided with the restoration members 62L, 62R, a bottom plate 20A of the main housing member 20 is formed with openings 63L, 63R, as shown in FIG. 11. These openings are used in forming the proximal ends of the restoration members as one with the left and right side wall sections 20L, 20R at the time of molding the main housing member 20.

The pricking device 60 having the above mentioned structure operates similarly to the pricking device 1. That is, a lancet unit 61, returned into the inside of the housing unit 3, is kept retained due to the lancet unit automatic lock mechanism to inhibit re-use of the lancet unit.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

The invention is claimed as follows:

1. A pricking device comprising:
a lancet unit integrally formed with a needle; and
a housing that houses said lancet unit for movement therein;
wherein said lancet unit includes
a unit body member movable within said housing in connection with a pricking operation to cause said needle to pop out of said housing; and
a resilient piece;
wherein said resilient piece includes a cam bump that may be engaged with a cam groove formed in said housing;
said resilient piece being integrally formed with said unit body member, said resilient piece storing a force of elasticity as said unit body member is moved in connection with said pricking operation, said cam bump engaging within a foremost part of said cam groove when said unit body member has returned into said housing after the pricking operation, said cam bump being kept engaged within said foremost part under said force of elasticity stored in said resilient piece to keep a state of inhibiting re-popping out of said needle;
said unit body member or said housing including a restoration member that stores therein another force of elasticity as said unit body member is moved in connection with said pricking operation, said restoration member causing said needle to be returned into the inside of said housing under said another force of elasticity after the pricking operation.

2. The pricking device according to claim 1, wherein
said restoration member is integrally formed with said unit body member, and wherein
said resilient piece and said restoration member being each a cantilevered substantially rectangular resilient plate integrally formed with and protruded from at least a side edge of said unit body member.

3. The pricking device according to claim 1 or 2, wherein
said housing is formed of a main housing member and a cover member, wherein
said main housing member being opened on one major side thereof and delimiting a lancet accommodating void portion for housing said lancet unit therein, and said cover member being combined with said main housing member in a manner of closing an opened part of said lancet accommodating void portion.

4. The pricking device according to any one of claims 1 to 3, wherein
said housing is integrally formed with a pair of housing halves and a thin-walled hinge interconnecting said housing halves by a synthetic resin material, and said housing halves delimiting a lancet accommodating void portion in which said lancet unit is housed.
